(19) United States
(12) Patent Application Publication
(10) Pub. No.: US 2018/0362619 A1
(43) Pub. Date: Dec. 20, 2018

(54) VARIANT ANTIBODIES FOR SITE-SPECIFIC CONJUGATION

(71) Applicant: BRISTOL-MYERS SQUIBB COMPANY, Princeton, NJ (US)

(72) Inventors: Paul O. SHEPPARD, Granite Falls, WA (US); Henrik ANDERSEN, Seattle, WA (US); Xiang SHAO, Milpitas, CA (US); Chetana RAO-NAIK, Walnut Creek, CA (US); Arvind RAJPAL, San Francisco, CA (US)

(73) Assignee: BRISTOL-MYERS SQUIBB COMPANY, Princeton, NJ (US)

(21) Appl. No.: 16/061,646

(22) PCT Filed: Dec. 20, 2016
(86) PCT No.: PCT/US2016/067663

§ 371 Date: Jun. 12, 2018

Related U.S. Application Data
(60) Provisional application No. 62/270,245, filed on Dec. 21, 2015.

(51) Int. Cl. C07K 16/08; A61K 47/68; C07K 16/28; C07K 16/30; A61P 35/00

(52) U.S. Cl. CPC C07K 16/087; A61K 47/6803; A61K 47/6849; A61K 47/6851; A61K 45/06; C07K 16/2875; C07K 16/30; A61P 35/00; A61K 2300/00; A61K 47/6889

(57) ABSTRACT

Variant antibodies having cysteine substitutions at selected positions in the Fc region can be conjugated via the thiol group of the substituted-in cysteine.

### CROSS-REFERENCE TO RELATED APPLICATIONS

This application claims the benefit under 35 U.S.C. § 119(e) of U.S. Provisional Application Ser. No. 62/270,245, filed Dec. 21, 2015; the disclosure of which is incorporated herein by reference.

### SEQUENCE LISTING

Incorporated herein by reference in its entirety is a Sequence Listing named “12642WOPCT_ST25.txt,” comprising SEQ ID NO:1 through SEQ ID NO:8, which includes nucleic acid and/or amino acid sequences disclosed herein. The Sequence Listing has been submitted herewith in ASCII text format via EFS-Web, and thus constitutes both the paper and computer readable form thereof. The Sequence Listing was first created using PatentIn 3.5 on Oct. 30, 2015, and is approximately 22 KB in size.

### BACKGROUND OF THE INVENTION

This invention relates to variant antibodies adapted for site-specific conjugation to a drug moiety and antibody-drug conjugates made from such variant antibodies and methods of making and using such variant antibodies and conjugates.

A type of anticancer agent that is generating strong interest is an antibody-drug conjugate (ADC, also referred to as an immunoconjugate). In an ADC, a therapeutic agent (also referred to as the drug, payload, or warhead) is covalently linked to an antibody whose antigen is expressed by a cancer cell (tumor associated antigen). The antibody, by binding to the antigen, delivers the ADC to the cancer site. There, cleavage of the covalent link or degradation of the antibody leads to the release of the therapeutic agent. Conversely, while the ADC is circulating in the blood system, the therapeutic agent is held inactive because of its covalent linkage to the antibody. Thus, the therapeutic agent used in an ADC can be much more potent (i.e., cytotoxic) than ordinary chemotherapy agents because of its localized release. For a review on ADCs, see Schrama et al. 2006.

The structure of an ADC can be represented generally as:

Ab-L-D (I)

where Ab is an antibody, L is a linker moiety, and D is a drug. A key step in the preparation of a conjugate is the formation of bond between the antibody and the linker-drug component, commonly referred to as the conjugation step. (Those skilled in the art will appreciate that formula (I) is simplified for clarity and that embodiments in which an antibody is conjugated to multiple linker-drug components or a linker carries multiple drugs can exist.)

A chemical reaction frequently used for the conjugation step is the Michael reaction, in which a thiol group on the antibody acts as a nucleophile and adds across a maleimide group in the linker-drug component:

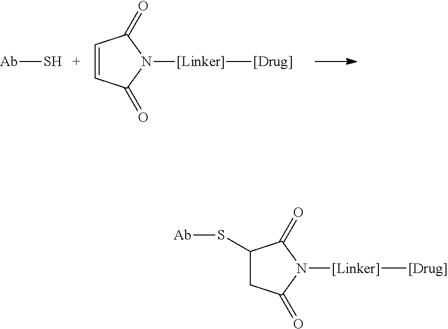

This reaction is advantageous because it proceeds readily under mild aqueous conditions.

An obstacle to using the Micahel reaction is the absence of reactive thiol groups in native antibodies. While antibodies possess numerous cysteine residues, their thiol groups are tied up in disulfide bonds and are unavailable to participate in a Michael addition. Hence, some modification of the antibody to introduce reactive thiol groups is necessary.

One way to introduce reactive thiol groups into an antibody entail treatment with 2-iminothiolane (Traut's reagent) to convert the —(CH₂)₄—NH₂ side chain of a lysine residue into a cysteine surrogate having a reactive thiol as shown below:

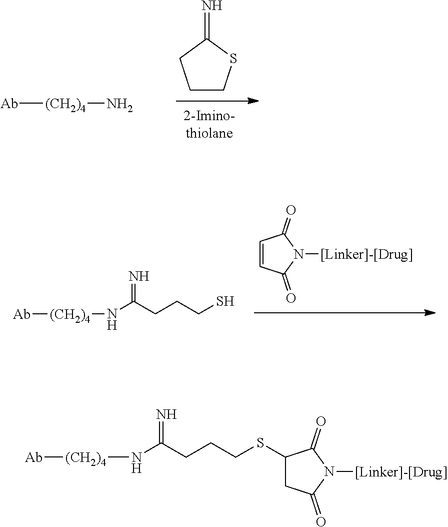

A limitation of this method is the lack of control over the number and location of the lysine residue(s) that are modified, resulting in a heterogeneous ADC product with varied antibody-drug ratios (DARs). For this reason, this method is referred to as a random conjugation method.

Another method to generate reactive thiol groups in an antibody is to reduce native disulfide bond(s), albeit at the risk of affecting antibody tertiary structure.

Yet another method to introduce reactive thiol groups into an antibody via site-specific mutations, in which an endogenous (native) amino acid is replaced by a cysteine. Examples of cysteine substitutions so purposed include Bhakta et al. 2016, Christie et al. 2016, Eigenbrot et al. 2009, Gao et al. 2015, Geierstanger et al. 2015 and 2016, Junutula et al. 2008 and 2010, Lloyd et al. 2015, Marquette et al. 2016, McDonagh et al. 2013, Shen et al. 2012, and Stimmel et al. 2000. The cysteine substitution may be accompanied by other modifications to the antibody, such as modification of its glycosylation state or other non-cysteine amino acid substitutions. The site of the cysteine substitution—i.e., the conjugation site—affects the stability and therapeutic activity of the ADC (Shen et al. 2012). Because the cysteines are introduced at predetermined positions, such conjugation is referred to as site-specific conjugation.

Site-specific cysteine substitutions for non-conjugation purposes such as “knob-into-holes” heterodimerization or modulating FcγR or FcRn binding, have also been disclosed. See, for example, Chamberlain et al. 2006 and 2012, Merchant et al. 1998, and Sondermann et al. 2007.

Other documents relating to substitutions in the Fc region include Lazar et al. 2007, 2008, and 2009 and Hansen et al. 2011.

Full citations for the documents cited herein by first author or inventor and year are listed at the end of this specification.

### BRIEF SUMMARY OF THE INVENTION

This invention provides novel site-specific cysteine substituted variant antibodies, in which an endogenous amino acid has been replaced with a cysteine in its Fc region, to provide a reactive thiol suitable for conjugation.

In a first embodiment, there is provided a variant antibody of the IgG isotype, comprising an Fc region having a cysteine substitution at one of positions 271, 289, 337, 340, 341, 343, 362, 402, 413, 414, 415, 419, 439, 440, and 441, the numbering of the positions being according to the EU index as in Kabat. Preferably, the cysteine substitution is at one of positions 271, 337, 340, 341, 343, 402, 413, 415, 419, 439, 440, and 441. (References to amino acid positions in an antibody Fc region employ numbering per the EU index as set forth in Kabat et al., “Sequences of proteins of immunological interest,” 5th ed., Pub. No. 91-3242, U.S. Dept. Health & Human Services, NIH, Bethesda, Md., 1991; hereinafter “Kabat.” The numbers themselves are referred to as EU, EU/Kabat, or EU as in Kabat numbers.)

In a second embodiment, there is provided an antibody-drug conjugate according to formula (II)

Ab(-L-(D)ₙ)ₘ (II)

wherein
Ab is a variant antibody of this invention,
L is a linker moiety,
D is a drug,
n is an integer from 1 to 30 (preferably 1 to 5, and more preferably 1), and
m is 1, 2, 3, 4, 5, or 6 (preferably 1 or 2),
   wherein Ab is bonded to L via a cysteine at one of positions 271, 337, 340, 341, 343, 402, 413, 415, 419, 439, 440, and 441 of the Fc region A, the numbering of the positions being according to the EU index as in Kabat. In a preferred embodiment, n is 1 and m is 1 or 2.

Linker L can be either of the cleavable or non-cleavable type. A cleavable linker relies on internalization of the ADC into a target cell and the action of a factor or agent present inside it to cleave the linker and release drug D. Where the linker contains a peptide group, it can be cleaved by an intracellular enzyme such as ones of the cathepsins, especially cathepsin B. Another enzyme that can be used to cleave a peptide-containing linker is legumain. Or, the linker can contain a disulfide group, with cleavage effected by disulfide exchange within the target cell, for example with glutathione. Or, the linker can be a hydrazine group, which can be cleaved at the lower pH conditions found inside intracellular bodies such as lysosomes, where ADCs are contained after internalization.

If linker L is of the non-cleavable type, it relies on degradation of the variant antibody to release the drug D. In such instances linker L remains attached to drug D and should be designed such that it does not interfere with the biological activity of drug D.

In a third embodiment, there is provided a method of treating cancer in a subject suffering from such cancer, comprising administering a therapeutically effective amount of an antibody-drug conjugate as described above.

### BRIEF DESCRIPTION OF THE DRAWING(S)

FIG. 1 shows schematically the architecture of an antibody, including the location of the Fc region (the C_{H}2 and C_{H}3 domains) where the site-specific cysteine substitutions of this invention are made.

FIG. 2 shows the consensus sequences for the Fc region of IgG antibodies, with the positions for site-specific cysteine substitution according to this invention highlighted.

FIG. 3 shows the positioning of the site-specific cysteine substitutions on a ribbon diagram of the C_{H}2 and C_{H}3 domains.

FIG. 4 shows schematically the application of orthogonal chemistry to the preparation of ADCs carrying two different payloads.

FIG. 5 shows a chromatographic trace used for calculating average DAR values in a conjugate.

### DETAILED DESCRIPTION OF THE INVENTION

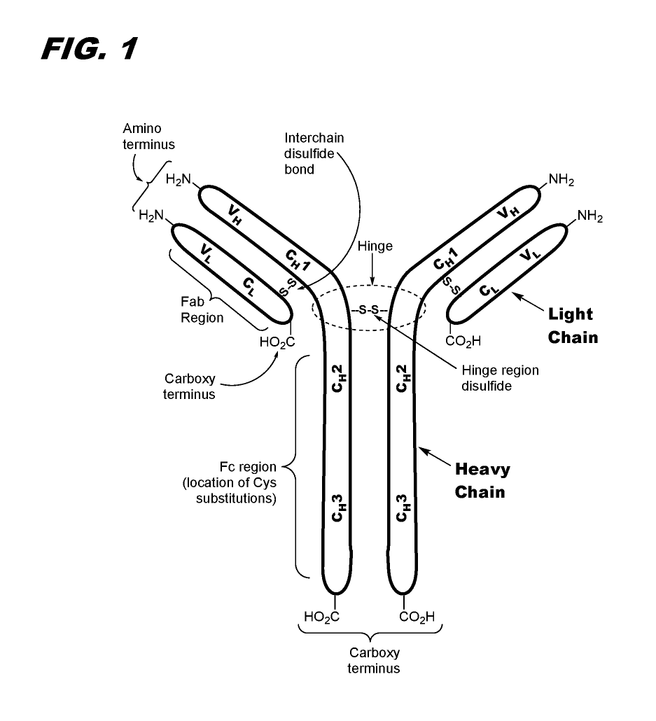

An antibody comprises two heavy (H) chains and two light (L) chains inter-connected by disulfide bonds. The light chains can be of the kappa or lambda type. Each heavy chain comprises a heavy chain variable region (V_{H}) and a heavy chain constant region comprising three regions or domains, C_{H}1, C_{H}2 and C_{H}3. The C_{H}2 and C_{H}3 regions are jointly referred to as the Fc region. The C_{H}2 and C_{H}3 regions are separated from the C_{H}1 region by an amino acid sequence referred to as the hinge region. Each light chain comprises a light chain variable region (V_{L} or V_{K}, according to whether the light chain is of lambda or kappa) and a light chain constant region comprising one single domain, C_{L}. Disulfide bridges connect each heavy and its partner light chain, the two heavy chains, and different locations within each heavy chain. The V_{H} and V_{L} regions can be further subdivided into regions of hypervariability, termed complementarity determining regions (CDRs), interspersed with more conserved framework regions (FRs). Each V_{H} and V_{L} comprises three CDRs and four FRs, arranged from amino- to carboxy-terminus in the following order: FR1, CDR1, FR2, CDR2, FR3, CDR3, and FR4. FIG. 1 shows schematically the architecture of an antibody. The variable regions contain a binding domain that interacts with an antigen. The constant regions may mediate the binding of the antibody to host tissues or factors, including various cells of the immune system (e.g., effector cells) and the first component (Clq) of the classical complement system. An antibody is said to “specifically bind” to an antigen X if the antibody binds to antigen X with a K_{D} of 5×10⁻⁸ M or less, more preferably 1×10⁻⁸M or less, more preferably 6×10⁻⁹ M or less, more preferably 3×10⁻⁹ M or less, even more preferably 2×10⁻⁹M or less. The antibody can be chimeric, humanized, or, preferably, human. Normally, an antibody is glycosylated at position N297 of the heavy chain, but the glycosylation type or extent (including elimination of any glycosylation) can be engineered, to extend antibody half-life, to enhance or reduce interactions with effector cells or the complement system, or to modulate some other property.

The C_{H}2 and C_{H}3 domains of the IgG-Fc domain typically consists of a total of 213 amino acids. Each of these amino acids contributes in different ways to the folding, stability, activity, and longevity of the molecule in vivo. To select which of these amino acids can be most efficiently targeted for a Cys substitution (mutation) that would be suitable for conjugation to a drug, many factors including residue accessibility, impact on proper protein folding, expression, and stability, were taken into account. For instance, introducing new Cys residues into a protein may cause competition for improper S—S (disulfide) bond formation with native Cys residues, resulting in a misfolded or unstable protein. Brute-force expression, purification, and characterization of proteins with substitution at each of the C_{H}2 and C_{H}3 amino acids requires significant resources, time, and expertise to accomplish. A triage of the 213 possibilities was used to reduce the number of possibilities requiring further evaluation at successively more resource intensive stages of evaluation.

To efficiently determine which subset of C_{H}2 and C_{H}3 amino acids were amenable to a Cys substitution, molecular modelling and sequence analysis were used as an initial screen to reduce the number of individual proteins to be produced, purified, and evaluated for conjugation. MOE molecular modelling tools were used to build models and collect structural statistics of all possible Cys mutation positions in the C_{H}2 and C_{H}3 domains of crystal structure 3WJJ (PDB reference code; see below). Each potential mutation position was evaluated for sufficient side chain surface exposure for conjugation accessibility (greater than 20%), lack of proximity to known antibody-attached carbohydrate, antibody dimeric chain, or CD32 binding regions (any atom within 4.5 Angstroms), distance from native Cys residues which might become involved in aberrant S—S bonds, and inspection for potential atom clashes with the native structure (destabilizing potential). Finally native residues such as A, G, and Pro which might have been eliminated due to size in the surface exposure analysis, were reviewed for potential inclusion as Cys mutant positions. Applying these measures, the original 213 positions were reduce to 89. This number of full length Ab proteins was deemed technically feasible to evaluate further by expression. Sequences representing these 89 mutations were then expressed in as described below and further evaluated for stability and/or conjugation efficiency to arrive at the specific Cys substitution sites of this invention.

Crystal structure 3WJJ can be downloaded from the Protein Data Bank (PDB). The terminal portion of the url for downloading the file is “rcsb.org/pdb/explore/explore.do?structureId=3wjj”, which can be converted to an active link by inserting “http://www.” in front of it.

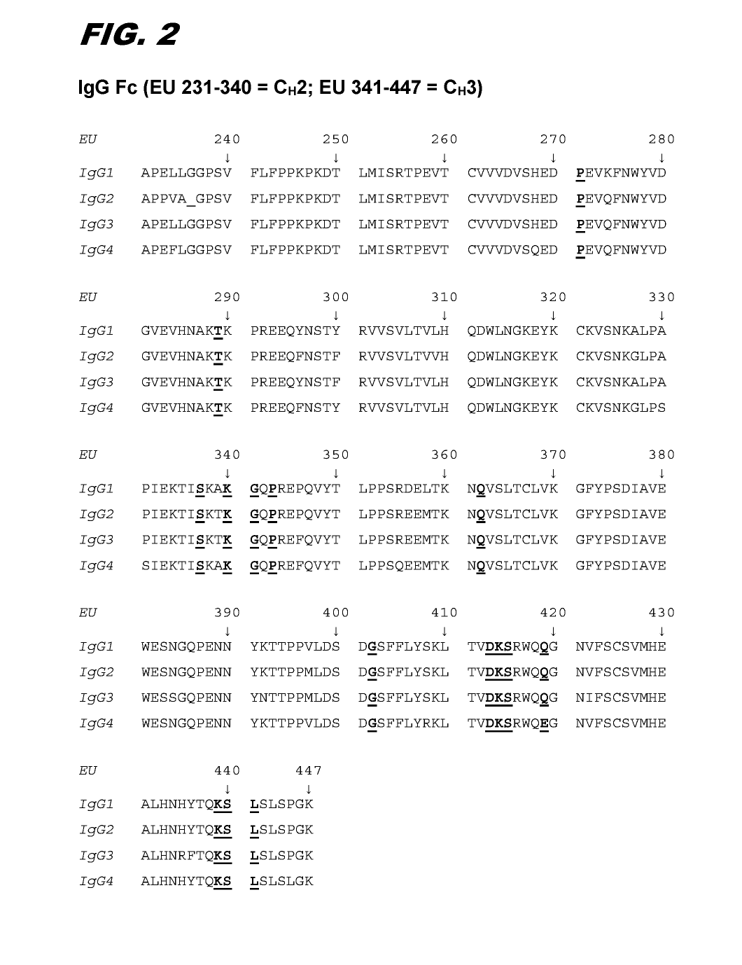

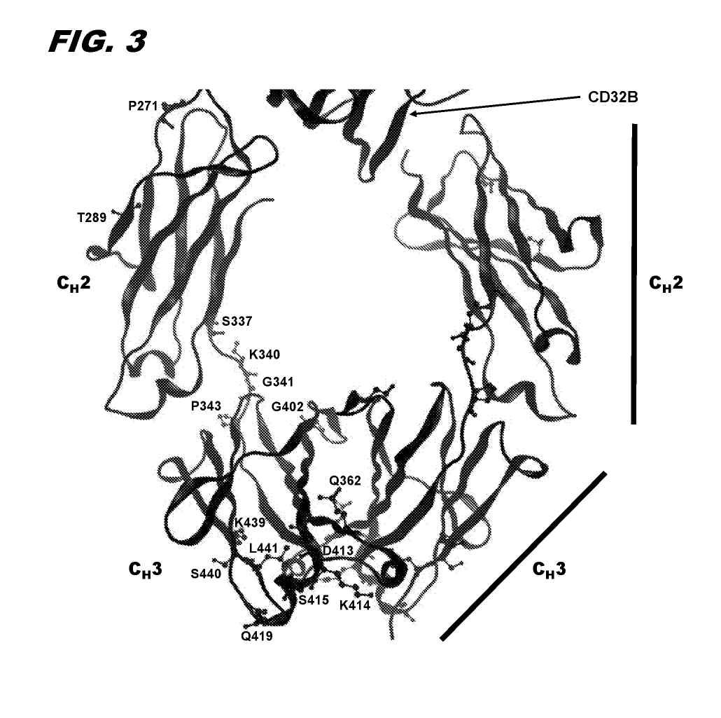

FIG. 2 shows the consensus amino acid sequences of the Fc regions of human IgG1, IgG2, IgG3, and IgG4 isotypes, with the sites of cysteine substitution according to this invention highlighted by bolding and underlining. The amino acids in the sequence are identified by EU/Kabat numbers, as is conventional for IgG Fc regions. In further keeping with convention, the substitutions can be referred to in shorthand format by listing in order the substituted-out original (endogenous) amino acid, the EU position number, and the substituted-in amino acid, as in P271C, T289C, etc. It is noteworthy that each of the substitution sites identified according to this invention is conserved across the IgG1, IgG2, IgG3, and IgG4 isotypes, excepting at EU position 419, which is glutamine (Q) in IgG1, IgG2, and IgG3 but glutamic acid (E) in IgG4. FIG. 3 shows the positioning of the site specific cysteine substitutions in the C_{H}2 and C_{H}3 domains, using a ribbon diagram based on the 3WJJ crystal structure.

The corresponding IgG1, IgG2, IgG3, and IgG4 Fc sequences are also provided in SEQ ID NO:1, NO:2, NO:3, and NO:4, respectively. SEQ ID NO:1 is annotated with a MISC_FEATURE remark at each site of cysteine substitution highlighted in FIG. 2, to provide a correlation between EU numbers and sequence listing numbering. SEQ ID NOs:2-4 do not have such annotations, but like correlations can be derived by reference to SEQ ID NO:1.

In a preferred embodiment, a variant antibody of this invention has a cysteine substitution at one of EU positions 337, 340, 341, and 343.

In another preferred embodiment, a variant antibody of this invention has a cysteine substitution at one of EU positions 413 and 415.

In yet another preferred embodiment, a variant antibody of this invention has a cysteine substitution at one of EU positions 439, 440, and 441.

In yet another embodiment, a variant antibody of this invention has a cysteine substitution at one of positions 271, 340, 341, 343, 402, and 439. Cysteine substitutions at such positions are advantageous in yielding conjugates with high DAR and/or low aggregation.

Cysteine substitution sites can be grouped according to physical proximity to each other. Roughly, according to the ribbon structure of FIG. 3, positions P271 and T289 can be placed in a Group A; positions 5337, K340, G341, P343, and G402 can be placed in a Group B; and positions Q362, D413, K414, S415, Q419, K439, S440, and L441 can be placed in a Group C. Variant antibodies of this invention can have plural cysteine substitutions. In such case, it is preferable to select substitutions that are spatially apart to reduce the likelihood of disulfide bond formation between them. For instance, combining a P271C (Group A) and a K340C (Group B) is recommended, as is combining a Q362C (Group C) and a G402C (Group B). However, it may be preferable to avoid combining Q362C and D413C (both Group C).

In one embodiment, each variant antibody heavy chain has one cysteine substitution, preferably at the same position in each chain (e.g., both have a P343C substitution or both have an S337C substitution). Such embodiment leads to an ADC with a theoretical DAR of two. In another embodiment, each variant antibody heavy chain has two cysteine substitutions (e.g., each has a P271C and a K340C substitution), leading to an ADC with a theoretical DAR of four. Variant antibodies in which each heavy chain has an even greater number of cysteine substitutions, or are not identically substituted, are also within the scope of this invention.

Human IgG antibodies occur in a number of allotypes (Jefferis and Lefranc 2009). For instance, the G1m3 allotype has E356 and M358 in the C_{H}3 region, instead of D356 and L358 as shown in FIG. 2. The scope of this invention is not limited to the allotypes shown in FIG. 2. Rather, human IgG antibodies having cysteine substitutions as taught herein but of other allotypes are also included within the scope of this invention.

A variant antibody of this invention can be of any of the IgG isotypes, but preferably is of the IgG1 or IgG4 isotype, and more preferably of the IgG1 isotype. The antibody can be chimeric, humanized, or, preferably, human. More preferably, the antibody is a human monoclonal antibody of the IgG1 or IgG4 isotype, and most preferably of the IgG1 isotype.

When an antibody is produced recombinantly, some of the heavy chain C-terminal chain lysine residues (amino acid 447 in FIG. 2) are often removed during the expression or purification steps by enzymes from the production host cell, leading to a heterogeneous product (both lysines present, one lysine removed, or both lysines removed). This heterogeneity is undesirable. To obtain a more heterogeneous product, both lysines can be intentionally removed, either by further enzymatic treatment of the initial product or by eliminating the codon for the C-terminal lysine from the nucleotide sequence used for recombinant expression. McDonough et al. 1992. Variant antibodies with the cysteine substitutions disclosed herein lacking heavy chain C-terminal lysine residues are also within the scope of this invention. Variant antibodies in which both the C-terminal glycine and lysine have been removed are also known and are included in the scope of this invention.

Variant antibodies of this invention can have, in addition to the cysteine substitutions disclosed herein, other types of alterations relative to the native type, including but not limited to those described following.

Antibodies of the IgG isotype have a glycosylation site at asparagine 297 (N297). The presence of the glycoside group may block access to certain amino acids on the antibody. In a well-known example, glutamine 295 (Q295) is not an amine acceptor substrate for the enzyme transglutaminase when the antibody is glycosylated at N297, but deglycosylation of the enzyme renders Q295 available as a transglutaminase substrate (Jeger et al. 2010). Similarly, some cysteine substitution sites according to this invention may be sterically obstructed, if only in part, by a glycoside group. In such instance removal of the glycoside group may make them more available for conjugation. Deglycosylation can be effected by post-translation treatment with an enzyme such as PNGase F (Peptide-N-Glycosidase F) to remove the glycoside group or by deleting the N297 glycosylation site with a site-specific substitution such as N297A. A similar effect might be achievable by, instead of removing a glycosyl group entirely, removing one or more saccharide units on it, thus changing its steric bulk.

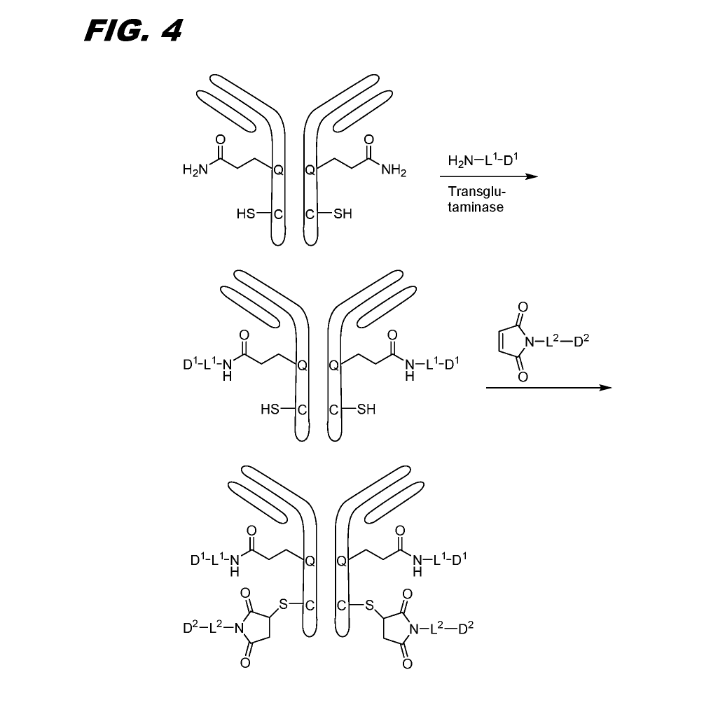

The methods of this invention for site-specific conjugation can be combined with other site-specific methods, to create plural orthogonal conjugation chemistries and enable the preparation of conjugates delivering two different drugs in a predetermined relative amount. The other site-specific conjugation method should be one involving chemistry other cysteine thiols, to create the orthogonality. This concept is illustrated in FIG. 4, with transglutaminase-mediated conjugation as the exemplary orthogonal conjugation chemistry. The illustrated antibody has, in its heavy chain a glutamine (Q) that is capable of acting as an amine receptor for transglutaminase and a cysteine substitution (C) according to this invention. Transglutaminase mediated conjugation of the glutamine with an amine donor H₂N-L¹-D¹, where L¹ is a first linker moiety and D¹ is a first drug, effects conjugation to provide an intermediate ADC carrying first drug D¹. Subsequent conjugation with a maleimide drug-linker compound

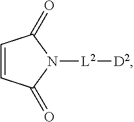

where L² is a second linker moiety and D² is a second drug that is different from drug D¹, effects conjugation to provide a final ADC carrying two different drugs, D¹ and D². (Those skilled in the art will appreciate that the order of the conjugation steps can be reversed.) Such an ADC is especially desirable in combination therapies, where two different drugs are used to attack a cancer simultaneously.

The transglutaminase-mediated conjugation illustrated in FIG. 4 is the direct, or one-step method. Alternatively, an indirect, or two-step method can be employed, as disclosed in Innate Pharma 2013.

The orthogonal conjugation chemistry used is not limited to transglutaminase coupling. Yet another conjugation technique involves introducing a non-natural amino acid into an antibody, with the non-natural amino acid providing a functionality for orthogonal conjugation chemistry. A non-natural amino acid can be introduced by engineering of the nucleotide sequence use to produce the antibody by recombinant expression, as taught in Tian et al., WO 2008/030612 A2 (2008). Non-natural amino acids can also be incorporated into an antibody or other polypeptide using cell-free methods, as taught in Goerke et al., US 2010/0093024 A1 (2010) and Goerke et al., Biotechnol. Bioeng. 2009, 102 (2), 400-416. If the non-natural amino acid p-acetylphenylalanine is introduced, the orthogonal conjugation chemistry can be oxime formation with a linker-drug compound having an NH₂ group. If the non-natural amino acid p-azidophenylalanine is introduced, the orthogonal conjugation chemistry can be “click chemistry,” in which the azido group reacts with a cyclooctyne group on the linker-drug compound to form an 1,2,3-triazole ring (Agard et al., J. Amer. Chem. Soc. 2004, 126, 15046; Best, Biochemistry 2009, 48, 6571).

Orthogonal conjugation chemistry can also be achieved by suitable modificaiton of the glycosyl group of the variant antibody. In one approach, a keto group is introduced into the glycosyl group, to serve as a conjugation site by oxime formation, as taught by Zhu et al., mAbs 2014, 6, 1. In another glycoengineering variation, an antibody's glycosyl group can be modified to introduce an azide group for conjugation by “click chemistry.” See Huang et al., J. Am. Chem. Soc. 2012, 134, 12308 and Wang, U.S. Pat. No. 8,900,826 B2 (2014) and U.S. Pat. No. 7,807,405 B2 (2010).

In addition to the cysteine substitution described above, a variant antibody of this invention can further have conservative substitutions at other amino acid positions. Such conservatively modified versions are included in the scope of this invention. A “conservative modification” or “conservative substitution” means, in respect of an antibody, the replacement of an amino acid therein with another amino acid having a similar side chain. Families of amino acids having similar side chains are known in the art. Such families include amino acids with basic side chains (lysine, arginine, histidine), acidic side chains (aspartic acid, glutamic acid), uncharged polar side chains (asparagine, glutamine, serine, threonine, tyrosine, cysteine, tryptophan), nonpolar side chains (glycine, alanine, valine, leucine, isoleucine, proline, phenylalanine, methionine), beta-branched side chains (threonine, valine, isoleucine), small side chains (glycine, alanine, serine), chain orientation changing side chains (glycine, proline) and aromatic side chains (tyrosine, phenylalanine, tryptophan). Plural conservative substitutions/modifications may be present. Preferably, where conservative substitutions are present, they are between 1 and 3 in number.

Antibodies that can be cysteine substituted according to this invention include those recognizing the following antigens: mesothelin, prostate specific membrane antigen (PSMA), CD19, CD22, CD30, CD70, B7H3, B7H4 (also known as O8E), protein tyrosine kinase 7 (PTK7), glypican-3, RG1, fucosyl-GM1, CTLA-4, and CD44. The antibody can be animal (e.g., murine), chimeric, humanized, or, preferably, human. The antibody preferably is monoclonal, especially a monoclonal human antibody. The preparation of human monoclonal antibodies against some of the aforementioned antigens is disclosed in Korman et al., U.S. Pat. No. 8,609,816 B2 (2013; B7H4, also known as 08E; in particular antibodies 2A7, 1G11, and 2F9); Rao-Naik et al., U.S. Pat. No. 8,097,703 B2 (2012; CD19; in particular antibodies 5G7, 13F1, 46E8, 21D4, 21D4a, 47G4, 27F3, and 3C10); King et al., U.S. Pat. No. 8,481,683 B2 (2013; CD22; in particular antibodies 12C5, 19A3, 16F7, and 23C6); Keler et al., U.S. Pat. No. 7,387,776 B2 (2008; CD30; in particular antibodies 5F11, 2H9, and 17G1); Terrett et al., U.S. Pat. No. 8,124,738 B2 (2012; CD70; in particular antibodies 2H5, 10B4, 8B5, 18E7, and 69A7); Korman et al., U.S. Pat. No. 6,984,720 B1 (2006; CTLA-4; in particular antibodies 10D1, 4B6, and 1E2); Vistica et al., U.S. Pat. No. 8,383,118 B2 (2013, fucosyl-GM1, in particular antibodies 5B1, 5B1a, 7D4, 7E4, 13B8, and 18D5) Korman et al., U.S. Pat. No. 8,008,449 B2 (2011; PD-1; in particular antibodies 17D8, 2D3, 4H1, 5C4, 4A11, 7D3, and 5F4); Huang et al., US 2009/0297438 A1 (2009; PSMA. in particular antibodies 1C3, 2A10, 2F5, 2C6); Cardarelli et al., U.S. Pat. No. 7,875,278 B2 (2011; PSMA; in particular antibodies 4A3, 7F12, 8C12, 8A11, 16F9, 2A10, 2C6, 2F5, and 1C3); Terrett et al., U.S. Pat. No. 8,222,375 B2 (2012; PTK7; in particular antibodies 3G8, 4D5, 12C6, 12C6a, and 7C8); Terrett et al., U.S. Pat. No. 8,680,247 B2 (2014; glypican-3; in particular antibodies 4A6, 11E7, and 16D10); Harkins et al., U.S. Pat. No. 7,335,748 B2(2008; RG1; in particular antibodies A, B, C, and D); Terrett et al., U.S. Pat. No. 8,268,970 B2 (2012; mesothelin; in particular antibodies 3C10, 6A4, and 7B1); Xu et al., US 2010/0092484 A1 (2010; CD44; in particular antibodies 14G9.B8.B4, 2D1.A3.D12, and 1A9.A6.B9); Deshpande et al., U.S. Pat. No. 8,258,266 B2 (2012; IP10; in particular antibodies 1D4, 1E1, 2G1, 3C4, 6A5, 6A8, 7C10, 8F6, 10A12, 10A12S, and 13C4); Kuhne et al., U.S. Pat. No. 8,450,464 B2 (2013; CXCR4; in particular antibodies F7, F9, D1, and E2); and Korman et al., U.S. Pat. No. 7,943,743 B2 (2011; PD-L1; in particular antibodies 3G10, 12A4, 10A5, 5F8, 10H10, 1B12, 7H1, 11E6, 12B7, and 13G4); the disclosures of which are incorporated herein by reference.

The subscript n in formula (II), repeated below, indicates the number of drugs D that bound to a linker. Often, one drug D is attached to each linker—i.e., n is 1—as exemplified by the approved ADCs MYLOTARG™, KADCYLA™, and ADCETRIS™. However, branched linkers can be used to so that multiple drugs D are attached to a single linker (i.e., n is greater than 1). For examples of branched linkers, see King et al. 2004 and Yurkovetsky 2015.

Ab(-L-(D)ₙ)ₘ (II)

A drug (therapeutic agent) for use in the conjugates of the variant antibodies of this invention typically is a cytotoxic agent that can kill a target cell. Examples include the following types of compounds and their analogs and derivatives:
(a) enediynes such as calicheamicin (see, e.g., Lee et al., J. Am. Chem. Soc. 1987, 109, 3464 and 3466) and uncialamycin (see, e.g., Davies et al., WO 2007/038868 A2 (2007); Chowdari et al., U.S. Pat. No. 8,709,431 B2 (2012); and Nicolaou et al., WO 2015/023879 A1 (2015));
(b) tubulysins (see, e.g., Domling et al., U.S. Pat. No. 7,778,814 B2 (2010); Cheng et al., U.S. Pat. No. 8,394,922 B2 (2013); and Cong et al., U.S. Pat. No. 8,980,824 B2 (2015));
(c) DNA alkylators such as analogs of CC-1065 and duocarmycin (see, e.g., Boger, U.S. Pat. No. 6,5458,530 B1 (2003); Sufi et al., U.S. Pat. No. 8,461,117 B2 (2013); and Zhang et al., U.S. Pat. No. 8,852,599 B2 (2014));
(d) epothilones (see, e.g., Vite et al., US 2007/0275904 A1 (2007) and U.S. Pat. No. RE42,930 E (2011));
(e) auristatins (see, e.g., Senter et al., U.S. Pat. No. 6,844,869 B2 (2005) and Doronina et al., U.S. Pat. No. 7,498,298 B2 (2009));
(f) pyrrolobezodiazepine (PBD) dimers (see, e.g., Howard et al., US 2013/0059800 A1(2013); US 2013/0028919 A1 (2013); and WO 2013/041606 A1 (2013)); and
(g) maytansinoids such as DM1 and DM4 (see, e.g., Chari et al., U.S. Pat. No. 5,208,020 (1993) and Amphlett et al., U.S. Pat. No. 7,374,762 B2 (2008)).

Preferably, the drug is a DNA alkylator, tubulysin, auristatin, pyrrolobenzodiazepine, enediyne, or maytansinoid compound, such as:

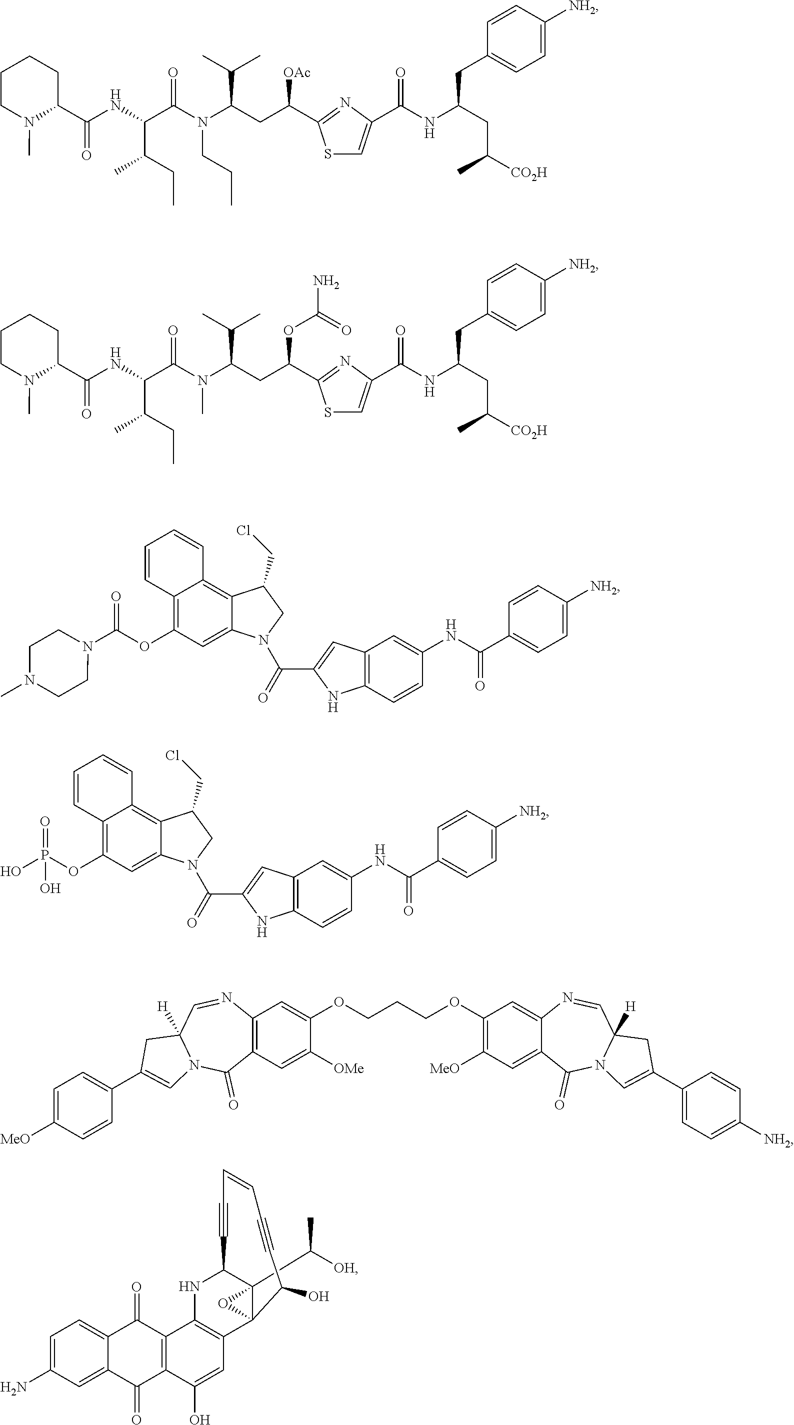

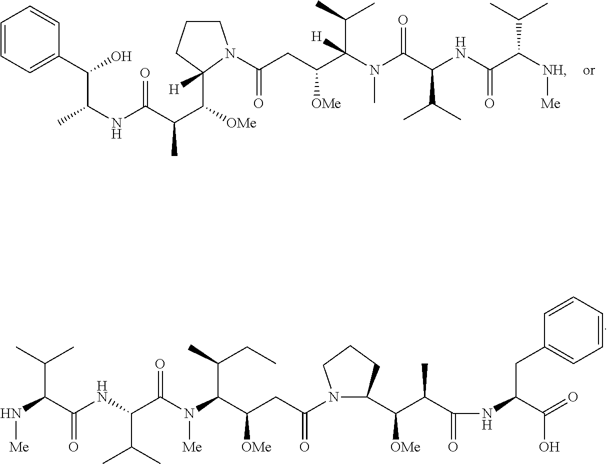

The functional group at which conjugation is effected is the amine (—NH₂) group in the case of the first five drugs above and the methyl amine (—NHMe) group in the case of the last two drugs.

To conjugate a drug to an antibody, a linker group is needed. The drug is combined with the linker to form a linker-drug compound, which is then conjugated to the adnectin. Thus, an antibody-drug conjugate can be prepared by reacting a variant antibody of this invention with a linker-drug compound wherein the linker has a maleimide group.

A preferred linker compound can be represented by formula (III):

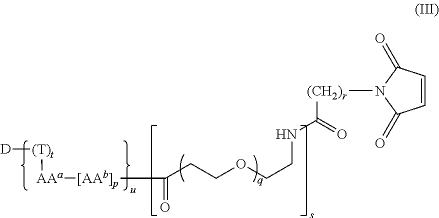

wherein
D is a drug;
T is a self-immolating group;
t is 0 or 1;
AA^{a} and each AA^{b} are independently selected from the group consisting of alanine, β-alanine, γ-aminobutyric acid, arginine, asparagine, aspartic acid, γ-carboxyglutamic acid, citrulline, cysteine, glutamic acid, glutamine, glycine, histidine, isoleucine, leucine, lysine, methionine, norleucine, norvaline, ornithine, phenylalanine, proline, serine, threonine, tryptophan, tyrosine, and valine;
p is 1, 2, 3, or 4;
u is 0 or 1
q is 2, 3, 4, 5, 6, 7, 8, 9, or 10;
r is 1, 2, 3, 4, or 5; and
s is 0 or 1.

In formula II, -AA^{a}-[AA^{b}]ₚ- represents a polypeptide whose length is determined by the value of p (dipeptide if p is 1, tetrapeptide if p is 3, etc.). AA^{a} is at the carboxy terminus of the polypeptide and its carboxyl group forms a peptide (amide) bond with an amine nitrogen of drug D (or self-immolating group T, if present). Conversely, the last AA^{b} is at the amino terminus of the polypeptide and its α-amino group forms a peptide bond with

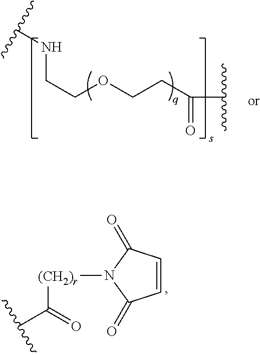

depending on whether s is 1 or 0, respectively. Preferred polypeptides -AA^{a}-[AA^{b}]ₚ- are Val-Cit, Val-Lys, Lys-Val-Ala, Asp-Val-Ala, Val-Ala, Lys-Val-Cit, Ala-Val-Cit, Val-Gly, Val-Gln, and Asp-Val-Cit, written in the conventional N-to-C direction, as in H₂N-Val-Cit-CO₂H). More preferably, the polypeptide is Val-Cit, Val-Lys, or Val-Ala. Preferably, a polypeptide -AA^{a}-[AA^{b}]ₚ- is cleavable by an enzyme found inside the target (cancer) cell, for example a cathepsin and especially cathepsin B.

If the subscript s is 1, drug-linker (I) contains a poly(ethylene glycol) (PEG) group, which can advantageously improve the solubility of drug-linker (I), facilitating conjugation to the antibody—a step that is performed in aqueous media. Also, a PEG group can serve as a spacer between the antibody and the peptide -AA^{a}-[AA^{b}]ₚ-, so that the bulk of the antibody does not sterically interfere with action of a peptide-cleaving enzyme.

As indicated by the subscript t equals 0 or 1, a self-immolating group T is optionally present. A self-immolating group is one such that cleavage from AA^{a} or AA^{b}, as the case may be, initiates a reaction sequence resulting in the self-immolating group disbonding itself from drug D and freeing the latter to exert its therapeutic function. When present, the self-immolating group T preferably is a p-aminobenzyl oxycarbonyl (PABC) group, whose structure is shown below, with an asterisk (*) denoting the end of the PABC bonded to an amine nitrogen of drug D and a wavy line ( [symbol] ) denoting the end bonded to the polypeptide -AA^{a}-[AA^{b}]ₚ-.

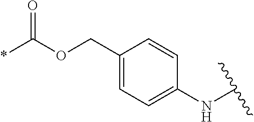 [SMILES: *Nc1ccc(COC(*)=O)cc1]

Another self-immolating group that can be used is a substituted thiazole, as disclosed in Feng, U.S. Pat. No. 7,375,078 B2 (2008).

Where the subscript u is 0, the linker does not contain either polypeptide -AA^{a}-[AA^{b}]ₚ- or self-immolating group T and is of the non-cleavable type.

The maleimide group in formula (III) serves as a reactive functional group for attachment to the reactive thiol in the antibody via a Michael addition reaction, as discussed above. Conjugation via the maleimide and a cysteine thiol in a variant antibody of this invention results in an antibody-drug conjugate according to formula (IV):

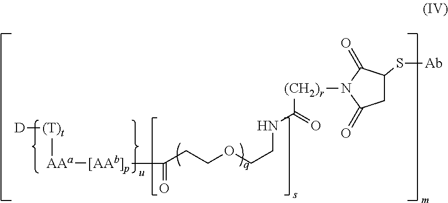

wherein
Ab is a variant antibody of the IgG isotype, comprising an Fc region having a cysteine substitution at one of positions 271, 337, 340, 341, 343, 402, 413, 415, 419, 439, 440, and 441, the numbering of the positions being according to the EU index as in Kabat;
D is a drug;
T is a self-immolating group;
t is 0 or 1;
AA^{a} and each AA^{b} are independently selected from the group consisting of alanine, β-alanine, γ-aminobutyric acid, arginine, asparagine, aspartic acid, γ-carboxyglutamic acid, citrulline, cysteine, glutamic acid, glutamine, glycine, histidine, isoleucine, leucine, lysine, methionine, norleucine, norvaline, ornithine, phenylalanine, proline, serine, threonine, tryptophan, tyrosine, and valine;
p is 1, 2, 3, or 4;
u is 0 or 1;
q is 2, 3, 4, 5, 6, 7, 8, 9, or 10;
r is 1, 2, 3, 4, or 5;
s is 0 or 1, and
m is 1, 2, 3, 4, 5, or 6 (preferably 1 or 2.

Antibody Ab is bonded to the linker-drug compound via the thiol group of a substituted-in cysteine (EU 271, 337, 340, 341, 343, 402, 413, 415, 419, 439, 440, or 441) by addition of the thiol across the maleimide double bond. The suffix m is 2 when the free thiol group in each of the substituted-in cysteines (one per heavy chain) is reacted with the maleimide group linker. Occasionally, only one of the thiol groups is reacted, resulting in an antibody-drug conjugate having only one linker-drug moiety attached—i.e., m is 1.

The practice of this invention can be further understood by reference to the following examples, which are provided by way of illustration and not of limitation.

### EXAMPLE 1

### Preparation of Variant Antibodies

Variant antibodies having cysteine substitutions according to this invention were prepared using an anti-mesothelin antibody designated as MSN-A and/or an anti-CD70 antibody designated as CD70-A. The heavy and light chain amino acid sequences of antibody MSN-A are given in SEQ ID NO:5 and SEQ ID NO:6, respectively. The heavy and light chain amino acid sequences of antibody CD70-A are given in SEQ ID NO:7 and SEQ ID NO:8, respectively.

The V_{H} and V_{K} fragments of MSN-A and CD70-A were cloned into a variety of mammalian expression vectors containing the constant regions for IgG1 antibody expression. These expression vectors also contained a puromycin or neomycin resistance gene to allow stable transfection for antibody production. Further, these expression vectors included mammalian display vectors that contained an intron and a trans-membrane domain after the heavy chain C_{H}3 domain, to allow both soluble and surface-bound antibody expression simultaneously from the same transfected cells.

An initial set 89 Cys substitutions in heavy chain C_{H}2 and C_{H}3 were chosen on the basis of their 3D structure, as discussed above. The DNA fragments containing these Cys mutations were synthesized and cloned into the mammalian expression vectors as described above to replace the wild type fragments. The molecular cloning for these constructs was achieved with in-fusion cloning technology or DNA ligation and E. coli transformation. The constructs containing these Cys substitutions were confirmed by DNA sequencing using the Sanger method.

These constructs were transfected into CHO-S cells and stable pools or clones were developed in culture media supplemented with puromycin and/or neomycin. The stable pools transfected with mammalian display vectors for the expression of variant antibodies with different Cys mutations were stained with PE-conjugated anti-human Kappa and APC-conjugated CD64 in FACS studies. Variants that retained CD64 binding, could be well expressed, and could be purified by Protein A were selected for further investigation.

### EXAMPLE 2

### Conjugation of Variant Antibodies

The following procedure is generally usable for the conjugation of the variant antibodies of this invention.

Variant antibodies were expressed in CHO cells and purified using protein A chromatography. A purified antibody were then treated with an excess (10-100 molar equivalents) of a reducing agent TCEP (tris(2-carboxyethyl)phosphine) at 37° C. for 0.5-3 hours in a buffered aqueous solution (pH 7-9). The TCEP was removed by passing the reduced variant antibody through a Sephadex G-25 column. The purified, reduced antibody was then treated with an excess (10-100 molar equivalents) of a disulfide formation reagent such as CuSO₄ (copper(II) sulfate), dhAA (dehydroascorbic acid), air, H₂O₂ (hydrogen peroxide), N—CS (N-chlorosuccinimide), or O₂ (molecular oxygen) at 4-37° C. for 0.5-24 h in a buffered aqueous solution (pH 4-9). The reoxidized antibody was purified by either ion exchange or size exclusion chromatography. The ratio of free thiols per antibody was estimated by determining the protein concentration from absorption of the protein solution at 280 nm, and the thiol concentration from reaction of the protein with DTNB (5,5′-dithiobis-(2-nitrobenzoic acid), Ellman's reagent).

After reduction and oxidation as described above, the antibody in buffered aqueous solution (pH 7-10) was treated with 1-10 molar equivalents of a drug-linker containing a cysteine-reactive functional group (maleimide, iodoacetamide, or similar reactive). Drug-linkers were typically dissolved in an organic solvent (DMSO, DMA, or similar), which was also added to the reaction mixture. The reaction was allowed to proceed for 1-4 h at 4-37° C. Afterwards, the antibody-drug conjugate was purified by ion exchange, size exclusion, protein A, or hydrophobic interaction chromatography, or a combination of multiple types of chromatography. Analytical testes such as SDS-PAGE, Western blots, HIC and Mass Spectrometry were carried out to confirm the attachment of the drug linker at the engineered position.

### EXAMPLE 3

### Conjugate Properties

Conjugates were prepared per the above procedure, using a maleimide-terminated linker with a tubulysin analog (see. e.g., Cheng et al., U.S. Pat. No. 8,394,922 B2 (2013) and Cong et al., U.S. Pat. No. 8,980,824 B2 (2013)) as the drug component, having a structure generally as shown below:

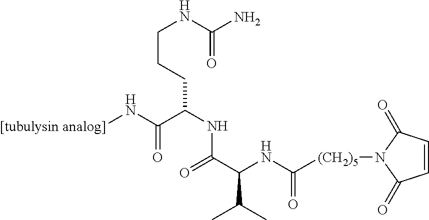

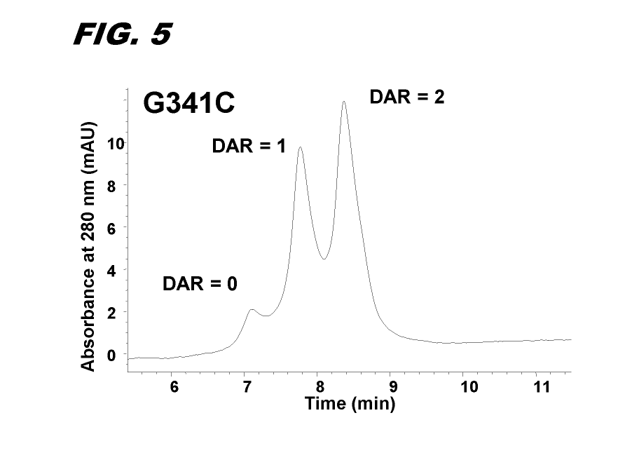

The conjugates were analyzed for their average DAR, using hydrophobic interaction chromatography and integrating the peak areas. A representative chromatographic trace, for an antibody with a G341C substitution, is shown in FIG. 5. Those skilled in the art will understand that the average DAR is a statistical average and that individual antibody molecules may have DAR values of zero, one, or two. Results are presented in Table I.

TABLE II
Average DAR of Conjugates

| Substitution | Average DAR |
|---|---|
| P271C | 0.89 |
| T289C | 1.21 |
| S337C | 0.56 |
| K340C | 1.39 |
| G341C | 1.46 |
| P343C | 1.38 |
| Q362C | 0.85 |
| G402C | 1.35 |
| D413C | 0.63 |
| K414C | 1.60 |
| S415C | 0.95 |
| Q419C | 1.20 |
| K439C | 1.29 |
| S440C | 1.18 |
| L441C | 0.69 |

### EXAMPLE 4

A preparation of a conjugate of antibody MSN-A having a P343C substitution and a tubulysin analog/linker compound per the previous example was tested in vitro against human gastric (stomach) cancer (N87) and human mesothelioma (H226) cancer cells. A ³H thymidine incorporation assay was used (Cheng et al., U.S. Pat. No. 8,394,922 B2 (2013)). The EC₅₀ values were 0.55 nM against N87 cells and 0.30 nM against H226 cells.

The foregoing detailed description of the invention includes passages that are chiefly or exclusively concerned with particular parts or aspects of the invention. It is to be understood that this is for clarity and convenience, that a particular feature may be relevant in more than just the passage in which it is disclosed, and that the disclosure herein includes all the appropriate combinations of information found in the different passages. Similarly, although the various figures and descriptions herein relate to specific embodiments of the invention, it is to be understood that where a specific feature is disclosed in the context of a particular figure or embodiment, such feature can also be used, to the extent appropriate, in the context of another figure or embodiment, in combination with another feature, or in the invention in general.

Further, while the present invention has been particularly described in terms of certain preferred embodiments, the invention is not limited to such preferred embodiments. Rather, the scope of the invention is defined by the appended claims.

### REFERENCES

Full citations for the following references cited in abbreviated fashion by first author (or inventor) and date earlier in this specification are provided below. Each of these references is incorporated herein by reference for all purposes.

Bhakta et al., US 2016/0130358 A1 (2016).

Chamberlain et al., US 2006/0173170 A1 (2006).

Chamberlain et al., EP 1817340 B1 (2012).

Christie et al., WO 2016/054315 A1 (2015).

Eigenbrot et al., U.S. Pat. No. 7,521,541 B2 (2009).

Gao et al., WO 2015/157595 A1 (2015).

Geierstanger et al., WO 2015/138615 A2 (2015).

Geierstanger et al., US 2016/0067351 A1 (2016).

Hansen et al., US 2011/0123440 A1 (2011).

Innate Pharma, “A New Site Specific Antibody Conjugation Using Bacterial Transglutaminase,” presentation at ADC Summit, San Fransisco, Calif., Oct. 15, 2013.

Jefferis and Lefranc, mAbs 2009, 1 (4), 1.

Jeger et al., Angew. Chem. Int. Ed. 2010, 49, 9995.

Junutula et al., Nature Biotechnol. 2008, 26 (8), 925.

Junutula et al., U.S. Pat. No. 7723485 B2 (2010).

King et al., U.S. Pat. No. 6,759,509 B1 (2004).

Lazar et al., US 2007/0237765 A1 (2007).

Lazar et al., WO 2008/092117 A2 (2008).

Lazar et al., US 2009/0010920 A1 (2009).

Lloyd et al., US 2015/155345 A1 (2015).

Marquette et al., US 2016/0008485 A1 (2016).

McDonagh et al., U.S. Pat. No. 8,455,622 B2 (2013).

McDonough et al., U.S. Pat. No. 5,126,250 (1992).

Merchant et al., Nature Biotechnol. 1998, 16, 677.

Schrama et al., Nature Rev. Drug Disc. 2006, 5, 147.

Shen et al., Nature Biotechnol. 2012, 30 (2), 184.

Sondermann et al., US 2007/0111281 A1 (2007).

Stimmel et al., J. Biol. Chem. 2000, 275 (39), 30445.

Yurkovetsly, US 2015/0306240 A1 (2015).

### Table of Sequences

TABLE II
Sequence Summary

| SEQ ID NO: | SEQUENCE DESCRIPTION |
|---|---|
| 1 | Human IgG1 Fc a.a. |
| 2 | Human IgG2 Fc a.a. |
| 3 | Human IgG3 Fc a.a. |
| 4 | Human IgG3 Fc a.a. |
| 5 | Antibody MSN-A heavy chain a.a. |
| 6 | Antibody MSN-A light (kappa) chain a.a. |
| 7 | Antibody CD70-A heavy chain a.a. |
| 8 | Antibody CD70-A light (kappa) chain a.a. |

What is claimed is:

1. A variant antibody of the IgG isotype, comprising an Fc region having a cysteine substitution at one of positions 271, 337, 340, 341, 343, 402, 413, 415, 419, 439, 440, and 441, the numbering of the positions being according to the EU index as in Kabat.

2. A variant antibody according to claim 1, wherein the cysteine substitution is at one of positions 337, 340, 341, and 343.

3. A variant antibody according to claim 1, wherein the cysteine substitution is at one of positions 413 and 415.

4. A variant antibody according to claim 1, wherein the cysteine substitution is at one of positions 439, 440, and 441.

5. A variant antibody according to claim 1, which is a human monoclonal antibody of the IgG1 isotype.

6. An antibody-drug conjugate according to the formula (II)
Ab(-L-(D)ₙ)ₘ (II)
wherein
Ab is a variant antibody according to claim 1,
L is a linker moiety,
D is a drug,
n is an integer from 1 to 30, and
m is 1, 2, 3, 4, 5, or 6,
wherein Ab is bonded to L via a cysteine at one of positions 271, 337, 340, 341, 343, 402, 413, 415, 419, 439, 440, and 441 of the Fc region A, the numbering of the positions being according to the EU index as in Kabat.

7. An antibody-drug conjugate according to claim 6, wherein drug D is a DNA alkylator, a tubulysin, an auristatin, a pyrrolobenzodiazepine, an enediyne, or a maytansinoid compound.

8. An antibody-drug conjugate according to claim 6, wherein n is 1 and m is 1 or 2.

9. An antibody-drug conjugate according to claim 6, wherein variant antibody Ab is a human monoclonal antibody of the IgG1 isotype.

10. An antibody-drug conjugate, having a structure according to formula (IV): 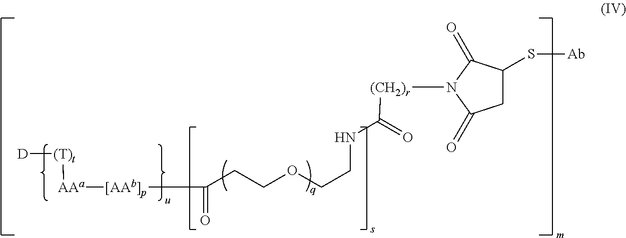
wherein
Ab is a variant antibody of the IgG isotype, comprising an Fc region having a cysteine substitution at one of positions 271, 337, 340, 341, 343, 402, 413, 415, 419, 439, 440, and 441, the numbering of the positions being according to the EU index as in Kabat;
D is a drug;
T is a self-immolating group;
t is 0 or 1;
AA^{a} and each AA^{b} are independently selected from the group consisting of alanine, β-alanine, γ-aminobutyric acid, arginine, asparagine, aspartic acid, γ-carboxyglutamic acid, citrulline, cysteine, glutamic acid, glutamine, glycine, histidine, isoleucine, leucine, lysine, methionine, norleucine, norvaline, ornithine, phenylalanine, proline, serine, threonine, tryptophan, tyrosine, and valine;
p is 1, 2, 3, or 4;
u is 0 or 1;
q is 2, 3, 4, 5, 6, 7, 8, 9, or 10;
r is 1, 2, 3, 4, or 5;
s is 0 or 1, and
m is 1, 2, 3, 4, 5, or 6.

11. An antibody-drug conjugate according to claim 10, wherein drug D is a DNA alkylator, a tubulysin, an auristatin, a pyrrolobenzodiazepine, an enediyne, or a maytansinoid compound.

12. An antibody-drug conjugate according to claim 10, wherein u is 1.

13. An antibody-drug conjugate according to claim 10, wherein antibody Ab is a human monoclonal antibody of the IgG1 isotype.

14. A method of making an antibody-drug conjugate, comprising reacting a variant antibody according to claim 1 with a linker-drug compound wherein the linker has a cysteine-reactive functional group.

15. A method according to claim 14, wherein the linker-drug compound has a structure according to formula (III): 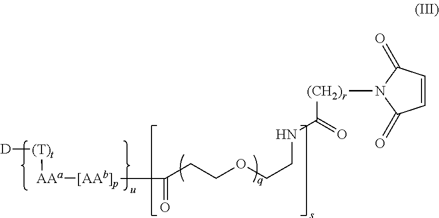
wherein
D is a drug;
T is a self-immolating group;
t is 0 or 1;
AA^{a} and each AA^{b} are independently selected from the group consisting of alanine, β-alanine, γ-aminobutyric acid, arginine, asparagine, aspartic acid, γ-carboxyglutamic acid, citrulline, cysteine, glutamic acid, glutamine, glycine, histidine, isoleucine, leucine, lysine, methionine, norleucine, norvaline, ornithine, phenylalanine, proline, serine, threonine, tryptophan, tyrosine, and valine;
p is 1, 2, 3, or 4;
u is 0 or 1;
q is 2, 3, 4, 5, 6, 7, 8, 9, or 10;
r is 1, 2, 3, 4, or 5; and
s is 0 or 1.

16. A method according to claim 15, wherein drug D is a DNA alkylator, a tubulysin, an auristatin, a pyrrolobenzodiazepine, an enediyne, or a maytansinoid compound.

17. A method according to claim 15, wherein u is 1.

18. A method according to claim 14, wherein the variant antibody is a human monoclonal antibody of the IgG1 isotype.

19. A method of treating a cancer in a subject suffering from such cancer, comprising administering to such subject a therapeutically effective amount of an antibody-drug conjugate according to claim 6.

20. A method of treating a cancer in a subject suffering from such cancer, comprising administering to such subject a therapeutically effective amount of an antibody-drug conjugate according to claim 10.


SEQUENCE LISTING (8 sequences)

SEQ ID NO: 1 - protein, 217 aa, Homo sapiens
  MISC_FEATURE (41)..(41): EU 271
  MISC_FEATURE (59)..(59): EU 289
  MISC_FEATURE (107)..(107): EU 337
  MISC_FEATURE (110)..(110): EU 340
  MISC_FEATURE (111)..(111): EU 341
  MISC_FEATURE (113)..(113): EU 343
  MISC_FEATURE (132)..(132): EU 362
  MISC_FEATURE (172)..(172): EU 402
  MISC_FEATURE (183)..(183): EU 413
  MISC_FEATURE (184)..(184): EU 414
  MISC_FEATURE (185)..(185): EU 415
  MISC_FEATURE (189)..(189): EU 419
  MISC_FEATURE (209)..(209): EU 439
  MISC_FEATURE (210)..(210): EU 440
  MISC_FEATURE (211)..(211): EU 441
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 217 aa, Homo sapiens
  MISC_FEATURE (6)..(6): Xaa is absent
APPVAXGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 217 aa, Homo sapiens
APELLGGPSVFLFPPKPKDTLMISRTPEVTCVVVDVSHEDPEVQFNWYVDGVEVHNAKTKPREEQYNSTFRVVSVLTVLHQDWLNGKEYKCKVSNKALPAPIEKTISKTKGQPREFQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 4 - protein, 217 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREFQVYTLPPSQEEMTKNQVSLTCLVKGFYPSDIAVEWESNGQPENNYKTTPPVLDSDGSFFLYRKLTVDKSRWQEGNVFSCSVMHEALHNHYTQKSLSLSLGK

SEQ ID NO: 5 - protein, 452 aa, Homo sapiens
  MISC_FEATURE (1)..(122): Variable region
  MISC_FEATURE (31)..(35): CDR1
  MISC_FEATURE (50)..(66): CDR2
  MISC_FEATURE (99)..(111): CDR3
  MISC_FEATURE (123)..(452): Constant region
QVHLVESGGGVVQPGRSLRLSCVASGITFRIYGMHWVRQAPGKGLEWVAVLWYDGSHEYYADSVKGRFTISRDNSKNTLYLQMNSLRAEDTAIYYCARDGDYYDSGSPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 214 aa, Homo sapiens
  MISC_FEATURE (1)..(107): Constant region
  MISC_FEATURE (24)..(34): CDR1
  MISC_FEATURE (50)..(56): CDR2
  MISC_FEATURE (89)..(97): CDR3
  MISC_FEATURE (108)..(214): Variable region
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 450 aa, Homo sapiens
  MISC_FEATURE (1)..(120): Variable region
  MISC_FEATURE (31)..(35): CDR1
  MISC_FEATURE (50)..(66): CDR2
  MISC_FEATURE (99)..(109): CDR3
  MISC_FEATURE (121)..(450): Constant region
EVQLLESGGGLVQPGGSLRLSCAASGFTFSIYAMSWVRQAPGKGLEWVSAISDSGGRTYFADSVRGRFTISRDNSKNTLSLQMNSLRAEDTAVYYCAKVDYSNYLFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 215 aa, Homo sapiens
  MISC_FEATURE (1)..(108): Variable region
  MISC_FEATURE (24)..(35): CDR1
  MISC_FEATURE (51)..(57): CDR2
  MISC_FEATURE (90)..(98): CDR3
  MISC_FEATURE (109)..(215): Constant region
EIVLTQSPGTLSLSPGERATLSCRASQSISSSYLAWYQQKPGQAPRLLIYGASSRATGIPDRFSGSGSGTDFTLTISRLEPEDFAVYYCQQYGSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC